(19) United States
(12) Patent Application Publication
(10) Pub. No.: US 2023/0288412 A1
(43) Pub. Date: Sep. 14, 2023

(54) COMPACT, ELECTRONICALLY READABLE POINT-OF-CARE IMMUNOASSAYS

(71) Applicant: The Government of the United States of America, as represented by the Secretary of the Navy, Arlington, VA (US)

(72) Inventor: David A. Kidwell, Alexandria, VA (US)

(21) Appl. No.: 18/119,276

(22) Filed: Mar. 8, 2023

Related U.S. Application Data
(60) Provisional application No. 63/318,391, filed on Mar. 9, 2022.

(51) Int. Cl. G01N 33/543

(52) U.S. Cl. CPC G01N 33/54388; G01N 33/5438; G01N 2033/0068

(57) ABSTRACT

The present invention provides for an analysis system and method that determines the presence of an analyte by generating electrical voltage and current. The intensity of the current is proportional to the amount of analyte present.

### PRIORITY CLAIM

The present application is a non-provisional application that claims the benefit of U.S. Provisional Application No. 63/318,391, filed on Mar. 9, 2022 by David A. Kidwell, entitled “COMPACT, ELECTRONICALLY READABLE POINT-OF-CARE IMMUNOASSAYS,” the entire contents of which are incorporated herein by reference.

This invention was made with government support by the U.S. Department of the Navy. The government has certain rights in the invention.

### BACKGROUND OF THE INVENTION

### Field of the Invention

The present invention relates to providing an electronically readable signal in lateral-flow immunoassays.

### Description of the Prior Art

Lateral-flow immunoassays (LFIAs) are common point-of-care (POC) tests for a wide variety of diseases and compounds. They are best known as home pregnancy tests and more recently as COVID 19 tests. LFIAs have the advantages of rapid results, no instrumentation required, and being self-timed due to the capillary flow of the analytes on the strip. LFIAs typically use colored particles as the visual label—most often colloidal gold particles or colored latex that are localized at a line due to a biological binding event (immunocomplex) of the various binding partners—for example antibodies or nucleic acids (Yu et al. 2011). The sensitivity of LFIAs is limited by the optical density of the gold label and this sensitivity is inadequate for some applications.

A number of researchers have proposed methods to increase the sensitivity of LFIAs. For example, Zhang, et al. have proposed gold nanoflowers as the labels (Zhang et al. 2019) whereas Yang, et. al. have proposed gold nanocages (Yang, Ozsoz, and Liu 2017). Basically, both systems just increase the size of the gold labels and thus show only a modest 2-3 fold increase in sensitivity over conventional labels. Labels other than gold show more promise in increasing sensitivity. For example, Linares, et al., following on the work of Rayev and Shmagel (Rayev and Shmagel 2008), has reviewed other labels and shown that carbon was about 10 fold more sensitive than gold (Linares et al. 2012).

There is only so much optical density a nanoparticle can have in the size regimen of about 50 nm and carbon, as shown by Linares et al., has reached that limit. One can increase the size of the nanoparticle label and thereby increase the optical density at the expense of performance in the LFIA, but even that has its limits as very large particles will not migrate up the strip. Alternatively, the size of the gold label can be increased after the biological binding event by forming complexes of complexes at a cost of increased steps for the user (Gao et al. 2017).

To increase the sensitivity of LFIAs, further amplification schemes have been proposed to increase the absorbance of the label after biological binding event by precipitation of metal at the site of the label. For example, Han et al. (Han, Choi, and Kwon 2016) have used the well-known autometallographic process of silver enhancement to enhance the colloidal gold labels detectability three fold. However, this chemical enhancement comes at the cost of increased complexity and user intervention in the LFIA system. Alternatively, the manufacturer must provide ways to delay the chemistry until after the biological events have occurred and deal with any manufacturing difficulties that complexity will entail. For example: Fu et al., have used two-dimensional structures for incorporation of multistep processes for improved sensitivity but at the cost of complex manufacturing (Fu et al. 2011).

To increase the sensitivity of LFIAs, other amplification schemes have been proposed. Building on much earlier catalytic work of Conyers and Kidwell (Conyers and Kidwell 1991), Kidwell has shown that appropriate dye systems and selected nanoparticles can enhance the sensitivity of LFIAs more than 1000 fold over gold labels by catalytically precipitating a dye at the site of the label. (Kidwell et al., “Catalytic Nanoparticles to Enhance the Sensitivity of Lateral Flow Immunoassays,” Nanotech 2019 Conference and Exposition, Boston, Mass., Jun. 17, 2019; (Kidwell 2018), (Kidwell 2021); (Kidwell 2022); and (Mulvaney et al. 2020))

Tominaga has used both enzymatic catalysts as well as chemical catalysts to localize a dye at the site of the label by manually applying a substrate after the biological event has occurred ((Tominaga 2017) and (Tominaga 2018)).

As was shown by the work of Mulvaney, et al., catalytic LFIA systems have a considerable sensitivity advantage over colorimetric labels. If the substrate of the catalyst is applied contemporaneously with the analyte solution, the catalyst will start developing the dye system (catalytic chemistry or substrate chemistry) thereby precipitating the dye along the strip as the capillary flow occurs. Thus, the substrate chemistry needs to be delayed until after the biological binding event and preferably after all the excess catalyst is wicked from the strip into the top absorbent pad. Delaying the substrate chemistry also has the advantage that the reagents need not be compatible to biology as often once the nanoparticle labels are localized by biology, they are hard to remove. Thus, unusual pH or strong oxidizing or reducing conditions can be used to optimize the catalyst activity rather than the biological activity of the binding partners.

As exemplified by Tominaga, a user could manually expose the developed strip to the catalytic chemistry, but this approach requires user interaction as well as separate packaging for the reagents. As exemplified by Fu, et al., the delay could be accomplished by having a two arm structure with one arm longer than the other and the length corresponding to a delay. Timing is limited as longer flow channels require more solution for filling. Additionally, this form often requires that the user of the LFIA place different solutions in different wells of the LFIA device and thus increases the complexity of handling as well as the need for several solutions. Another approach is to provide a diffusion barrier as exemplified by Kidwell, where timing is determined by the porosity of the barrier (Kidwell 1993) and (Kidwell 1994). Timing can be varied over a wide range as the diffusion can be adjusted due to pore size and pore density of the barrier. This would again require two wells—one for application of the sample and another for application of the solution to dissolve the reagents—although these solutions could be identical. An alternative to a diffusion barrier is a dissolving barrier as exemplified by Lutz et al. (Lutz et al. 2013). This also has the advantage that the timing can be varied but it exposes the strip and catalyst to whatever the material is used to produce the barrier. For example, if dissolving glucose is used as the barrier, the strip and catalyst would be exposed to a saturated solution of glucose. Another alternative is varying the pressure on individual wells as exemplified by Lawrence, et al. (Lawrence et al. 2019). The method of Lawrence et al. could be thought of as equivalent to having variable diffusion barriers where the diffusion is controlled by squeezing a sponge, which changes the pore size and diffusion rate.

Colorimetric labels have disadvantages in sensitivity, which is partly mitigated using catalytic particles. However, there is an ongoing need in the art for devices and methods that provide quantitative results yet retain the ease of use of LFIAs. For example, such devices may allow medical practitioners to diagnose, monitor, and manage a variety of conditions such as disease burden or treatment progress without an extensive infrastructure. A concrete example is seen in the work of Phillips and Krum (Phillips and Krum 1998) who showed that accurate quantitation of up to ten different cytokines would form patterns that could distinguish normal individuals from those with a virial disease or hay fever. Visual comparison of line density is often not sufficient to be quantitative as shown by Pickering, et al. (Pickering et al. 2021).

A large number of applications have focused on the idea of using digital equipment for quantitation. For example, (Ehrenkranz 2015). One solution is to read the LFIA using a photoreflective reader, as exemplified by the a commercial Digital Pregnancy Test, which contains electronics that measure the color density of the captured line and control line using an LED light source and photodetector. It is sold as First Response Gold Digital Pregnancy Test and is described in Nazareth et al., (Nazareth et al. 2010) and subsequent patents and patent applications. More recent implementations for the use of cameras in a smart phone or scanners is reviewed by Mulvaney, et al. (Mulvaney, et al.). Photographic reading has limitations on lighting levels and the presence of background on the strip that makes the lines difficult to observe.

In a conventional; fuel cell as reviewed by Winter and Brodd (Winter and Brodd 2004), the electrodes must have a catalyst present to rapidly oxidize and reduce the fuels when the fuel cell is operated at temperatures less than about 200° C. For a Polymer Electrolyte Membrane Fuel Cell (PEMFC) based on hydrogen as the fuel, platinum is the common catalyst. In the PEMFC platinum is often employed at about 100 μg/cm² on the anode to catalyze the hydrogen oxidation. Also in the PEMFC, platinum is used on the cathode at about 500 μg/cm² to catalyze oxygen reduction.

There is an ongoing need in the art for devices and methods that measure the binding events in an LFIA directly through electronic means without complex electronics.

### BRIEF SUMMARY OF THE INVENTION

The present invention provides a system and method to directly read the binding events in a LFIA. Using catalytic particles as the detection reagent allows use of their catalytic properties to transform the LFIA system from a visual system into one based on fuel cells that output a voltage and current. We term this system the fuel-cell Lateral-Flow ImmunoAssay or fcLFIA. As the current generated in the fcLFIA is proportional to the amount of catalyst which is proportional to the binding of the catalyst at the electrode due to biology, the amount of binding and hence target molecules can be measured directly. A further advantage of the fcLFIA platform is that as current can be readily measured electronically; the sensitivity can be increased over colorimetic-based LFIAs.

It is a goal of the present invention to provide quantitation of the LFIA without greatly affecting the ease of use of conventional LFIAs. It is another goal of the present invention to increase sensitivity over colorimetric LFIAs. It is an additional goal of the present invention to be able to transmit and store the values obtained and performance of the platform either locally or through a connection to a remote site for further analysis of the results as well as provide a historic record.

These and other features and advantages of the invention, as well as the invention itself, will become better understood by reference to the following detailed description, appended claims, and accompanying drawings.

### BRIEF DESCRIPTION OF THE DRAWINGS

FIG. 1 is a diagram showing a sandwich-type LFIA to demonstrate the steps in a LFIA process.

FIG. 2 is stylized view of an electrode system and a typical LFIA support strip.

FIG. 3 shows one embodiment of a LFIA cartridge that holds the electrode system and the reader that makes contact to the electrode system.

FIG. 4 shows electrodes and a drawing of the assembled fcLFIA system including placement of the components.

FIG. 5 is another embodiment of the fcLFIA cartridge that uses capillary action to control fluid flow.

FIG. 6 is another embodiment of the fcLFIA cartridge using a homogeneous assay rather than requiring separation steps.

FIG. 7 is a schematic of the reader as an interface to a desktop computer.

FIG. 8 is a simplified, block diagram of the reading electronics as depicted in FIG. 7.

FIG. 9 is a graph showing how fluid flow can be detected due to the voltage change on the various electrodes.

FIGS. 10A-C shows representative voltage and current curves taken in Example 5. The graph in FIG. 10A shows the total data set. The graph in FIG. 10B is an expanded version after hydrogen fuel was added for stressing each part of the fuel cell. The graph in FIG. 10C is the power calculated from stressing only the fuel cell before (Middle #1) and after (Middle #2-5) adding the hydrogen fuel.

FIGS. 11A-B show data taken with various resistors in series and measuring the resultant currents and voltage. FIG. 11A is a graph of the currents during the stressing. FIG. 11B is a plot of log of the load resistance vs. measured average current for the first four loadings in FIG. 11A.

FIGS. 12A-B show data taken with various resistors in series and measuring the resultant currents and voltage. The maximum power from this series is displayed in the chart. FIG. 12A is a graph of the currents during the stressing. FIG. 12B is a plot of log of the load resistance vs. measured average current for the first four loadings in FIG. 12A.

### DETAILED DESCRIPTION OF THE DRAWINGS

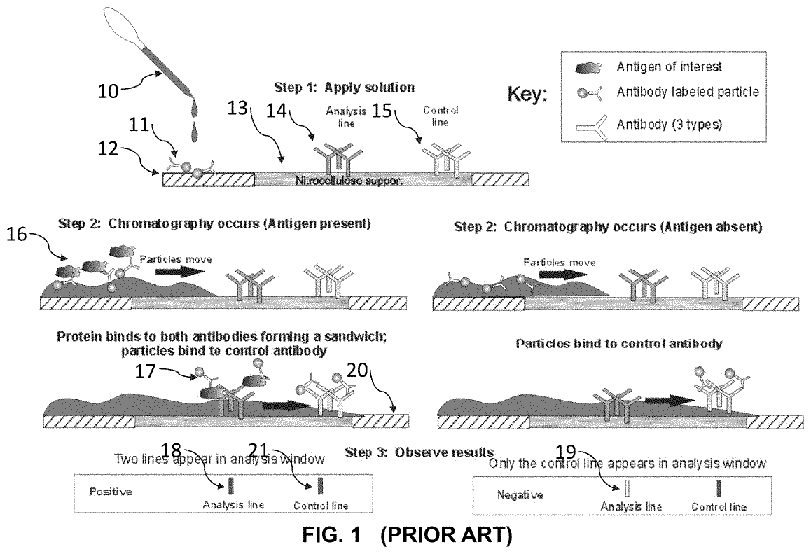

FIG. 1 shows the steps in a typical lateral-flow assay depicting a sandwich type assay. Other types are known in the art and can these may be employed with the fcLFIA system. In Step 1, a test solution is applied to a conjugate pad on the strip. The test solution 10 dissolves and mobilizes antibodies bound to colored nanoparticles 11 on the conjugate pad 12. The mobilized antibody-nanoparticles suspension travels through the conjugate pad into the nitrocellulose substrate 13 through capillary action. As the suspension travel up the nitrocellulose strip, it comes into contact with immobilized antibodies 14, 15 placed in lines across the nitrocellulose strip. If an antigen 16 is present in the test solution, that antigen binds to both the antibodies on the antibody-nanoparticle construct and the antibodies that have been immobilized on the nitrocellulose strip 17. Due to the color of the nanoparticles, this forms an analysis 18 line at the site of immobilization. If no antigen is present in the test solution, then no line is formed 19. Finally, excess test liquid and mobilized antibody-nanoparticles are wicked into the absorption pad 20. The control line 21 may comprise anti-antibodies to the antibodies on the antibody-nanoparticles 11. The control line 15 captures excess antibody-nanoparticles 11 not bound to line 14. The control line demonstrates to the user that the antibody-nanoparticles 11 have been mobilized and are being wicked up the strip. Normally for sandwich assays, a large excess of antibody-nanoparticle constructs are used relative to the amount of antigen in the test solution. The nanoparticles are colored and rarely are completely captured by the two antibody lines. Unless a chaser liquid is used, which is almost never done as it increases the steps the operator of the LFIA needs to perform, the test solution will provide a slightly colored background to the strip. Any background near the capture lines lowers the sensitivity of the assay as differences in color between the background and the lines is what is observed.

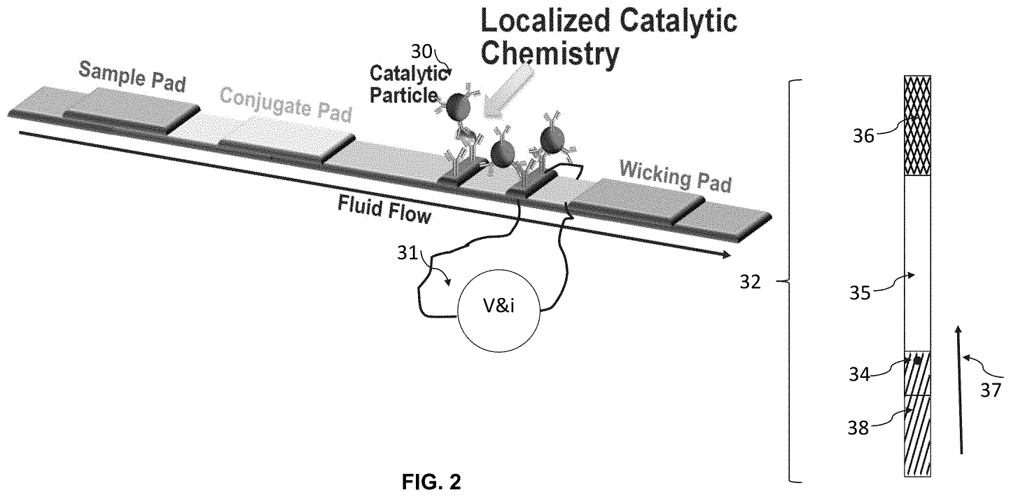

FIG. 2 displays a simplified concept for the instant invention. Unlike the prior art in FIG. 1, FIG. 2 depicts that the instant invention uses catalytic particles 30 as the labeling species and measures current and voltage 31 at the electrodes. A schematic of the active strip 32 comprising a conjugate pad 38, dried antibody-nanoparticle conjugate 34, a nitrocellulose membrane 35, and an absorption pad 36 is also depicted. The fluid flow is depicted with arrow 37. The active strip 32 is mainly for controlling fluid flow as no biological binding occurs on this strip, unlike with the prior art.

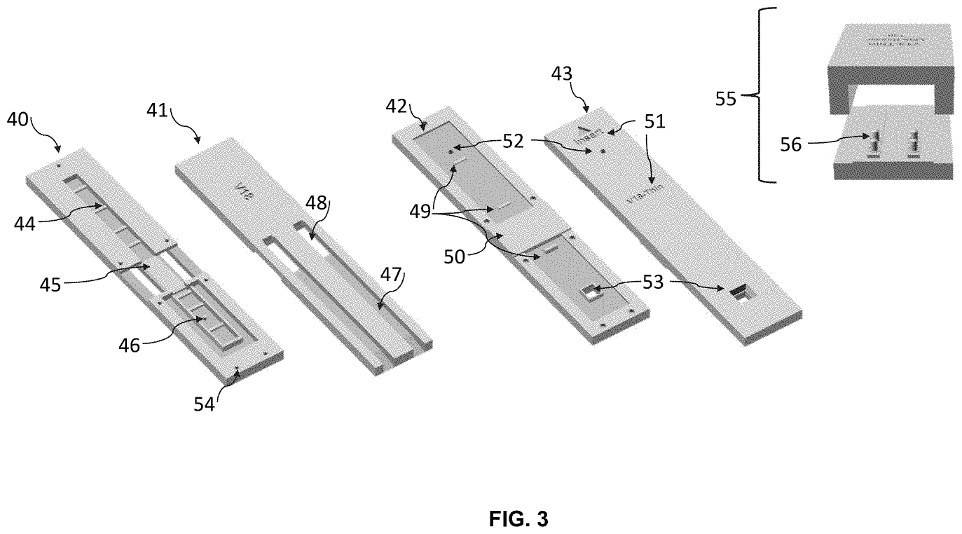

FIG. 3 is a picture of one embodiment of the fcLFIA cartridge, which is comprised of two pieces containing all the necessary chemistry and electrodes. The top view of the bottom piece 40 contains a slot and support areas 46 for the conjugate pad, the nitrocellulose strip support area 45, and the absorption pad support 44. The underside view of the bottom piece 41 contains guide slots 47 for insertion of the completed fcLFIA into the reader. The slots compress the pogo pins that make contact to the electrodes though holes 48 in the bottom piece. The bottom of the top piece 42 contains a plurality of supports 49 that compress and hold the active strip in the channel comprising the support areas 44, 45, and 46. Shelf 50 provides the backing and compression for the electrode assemblage. The top side of the top piece 43 contains user helpful markings 51, and air vent 52 and the well 53 for placement of the sample test solution. Pins 54 or other entities known in the art are used to hold the top and bottom layers together. The fcLFIA cartridge is inserted into the reader 55 (exploded in the drawing), which makes electrical contact from the bottom of the completed fcLFIA cartridge using pogo-pins 56.

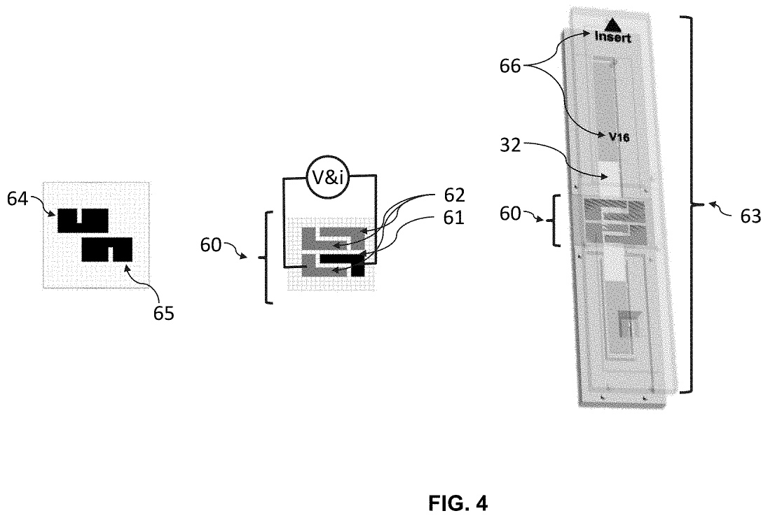

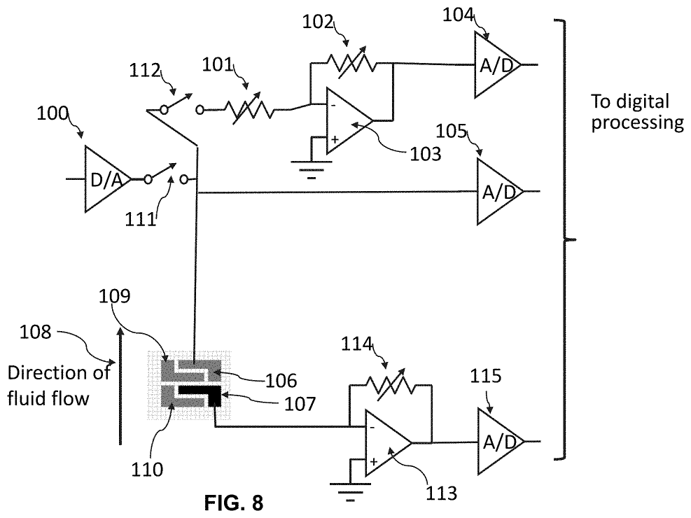

FIG. 4 depicts a schematic of one embodiment of the printed electrode assembly 60. The plurality of working electrodes 62 has at least one counter electrode 61. The current and voltage is read between each pair of electrodes through techniques known in the art and exemplified in FIGS. 8 & 9. A drawing of an assembled fcLFIA cartridge containing the active strip 32 and electrode assembly 60 was made with a semi-transparent top to demonstrate the placement of the various components. The top-down version shows back of the electrode assembly 60, which comprises a transparent support and printed carbon electrodes, generally printed on transparent polycarbonate plastic. The top being semi-transparent in this embodiment, the carbon electrodes placement and contact with the active strip 32 is evident. The electrodes are facing down. The cartridge 63 may have embossing 66 or other writing present that is useful to the user and can identify the cartridge to the reading system. Electrodes 64 and 65 are on a two electrode assembly that can replace the multiple electrode assembly 60. A two electrode assembly is useful for comparing catalytic activities of various preparations of nanoparticle conjugates with the fcLFIA cartridge.

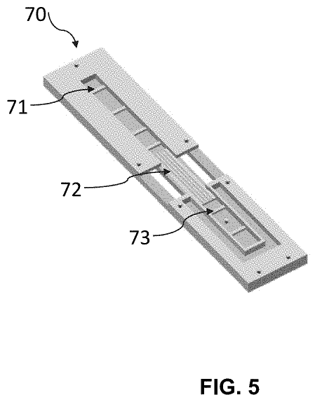

FIG. 5 depicts an embodiment of the bottom piece 70, which comprises a plurality of grooves 72, which replace the nitrocellulose media for wicking the test fluid into the absorption pad placed in well 71. The conjugate pad is placed in well 73. To seal the grooves, the electrode assembly is used as the top to form capillaries for fluid flow. In this manner, the test fluid is wicked into and through the grooves by capillary action and comes into close contact with the electrodes in the electrode assembly.

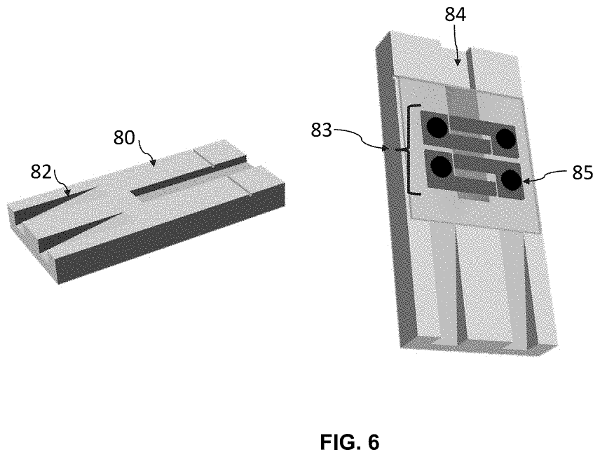

FIG. 6 depicts another embodiment of the fcLFIA system. The holder 80 contains ramps 82 to allow inserting into the reader, which compress the pogo pin contacts. The contacts make contact to the additional carbon electrodes 85 printed on the back of the electrode assembly 83. The test fluid is contained in the well 84. In one embodiment, holes are laser drilled through the plastic backing of electrode 60 from FIG. 4 and into the electrodes on the front. The holes are filled with carbon during printing of the back contacts to make electrical contact with electrodes on the back. One of the electrodes may be the ground electrode and the others being the working electrodes. A common ramp and pin spacing allows the same reader to work with all three embodiments. Because only catalyst that is in intimate contact with the electrodes can generate current, the fcLFIA system does not require a separation step to make the reading. Thus, a homogenous system can be developed as depicted in FIG. 6.

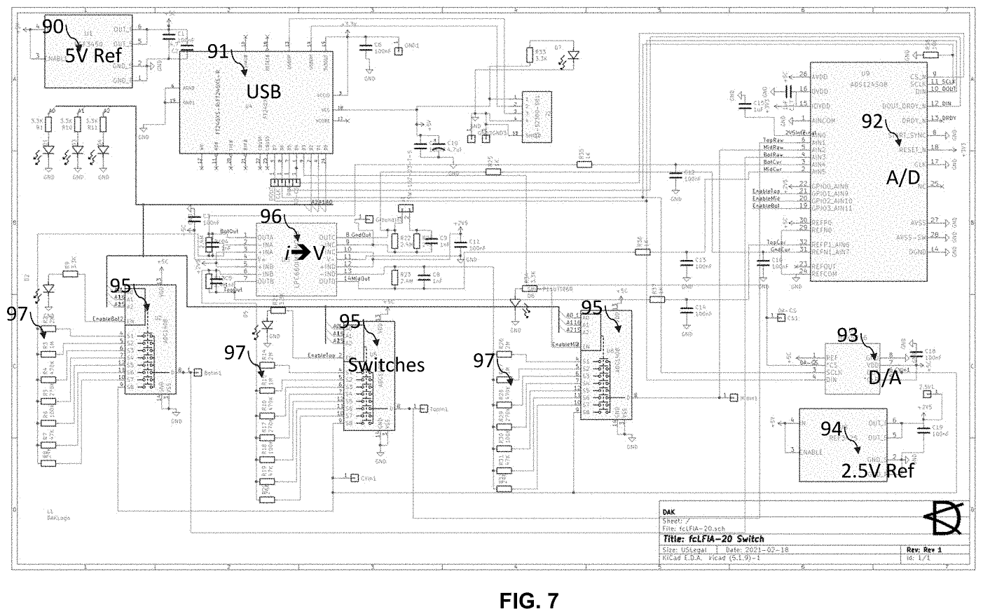

FIG. 7 shows the schematic for electrical interrogation of the fcLFIA electrode assembly through the pogo pin contacts. The major components are labeled. In one embodiment, power is supplied though a USB connector to the USB interface 91, which also provides two-way communication though the USB interface into a desktop computer. The raw 5V USB power is regulated into 5V and 2.5 V with linear regulators 90 & 94. Voltage can be applied to selected electrodes on the electrode assembly through the D/A 93 and the digital switches. The digital switches 95 can also apply a load through various resistors 97 to the individual electrodes in the electrode assembly. Current is measured with a current to voltage OP AMP circuit 96, known in the art. All voltages are converted to digital values with the 24 bit A/D 92 for transmission to the desktop computer through the USB interface 91. This embodiment can read voltages as +/−2.5V. By modifying voltage references 90 & 94, other voltages ranges can be accessed. A simplified block diagram of the electronic interface is shown in FIG. 8.

FIG. 8 depicts a simplified block diagram of the electronic interface highlighting only one of the number of circuits. The fuel cell is considered the electrochemistry occurring between a working electrode 106 and the ground electrode 107. The electrodes 106, 109, and 110 are the working electrodes. The fluid flow in the assembled device is indicated by arrow 108. Electrode 106 is conveniently titled the Top Electrode. In this embodiment, electrode 107 is the common ground for all the electrodes. Multiple grounds may be used if isolation of individual cells in the fuel cell is desired. Electrode 109 is conveniently titled the Middle Electrode, which is not considered in the simplified diagram. Electrode 110 is conveniently titled the Bottom Electrode, which is not considered in the simplified diagram. The open-circuit voltage on electrode 106 is measured by the A/D 105 when the two switches 111 and 112 are open. OP amp 103 provides a virtual ground to the variable resistor 101, which loads the fuel cell if switch 112 is closed. Current is measured in two ways: (1) OP amp 103 provides a current to voltage conversion, whose sensitivity is defined by resistor 102. The voltage output by OP amp 103 is converted to a digital value by A/D 104. (2) OP amp 113 provides a virtual ground to the ground electrode and is connected at all times in this embodiment. OP amp 113 also provides a current to voltage conversion, whose sensitivity is defined by resistor 114. The voltage output by OP amp 113 is converted to a digital value by A/D 115. Having the currents measured both ways is not completely necessary but it provides a check on the operation of the system as the currents should be opposite sign and similar values. D/A 100 can impress a voltage on the working electrode 106, which can be used for electrolysis to generate fuels or place a load on the fuel cell to measure current. When D/A 100 is active, the switch 112 is typically open as the current should be supplied by the fuel cell and not the D/A. The amount of current provided by the fuel cell due to the catalyst being present can be measured in two ways: (1) by impressing a voltage with D/A 100 and measuring a current with OP amp 113, or (2) varying the load resistance with resistor 101 and measuring currents with both OP amps 103 and 113. When measuring the current with OP amp 113 only (as switch 112 is open), stray, background currents can be determined by OP amp 103.

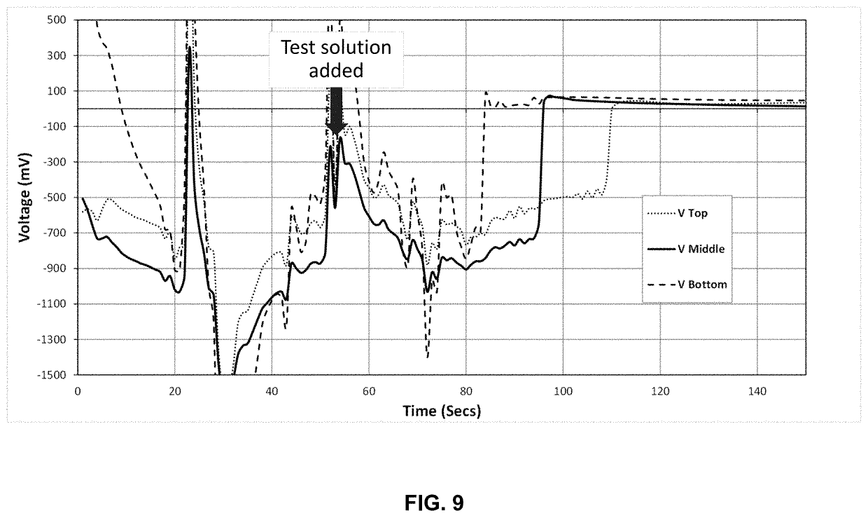

FIG. 9 is a graph showing the initial voltage levels of a sample run conducted as in Example 5. In one embodiment of the system, as the test sample fluid flows up the strip and connects each electrode to the ground, the open-circuit voltage goes from floating to near zero. Generally, the floating voltage is more negative than −500 mV. Note that the order of electrode response from Bottom [symbol] Middle [symbol] Top (as defined in FIG. 8) mirrors the fluid flow. This data provides some indication that the system is responding as expected—exclusive of a typical control line on the LFIA in the prior art, shown in FIG. 1. The fcLFIA may also have a control line as a separate electrode.

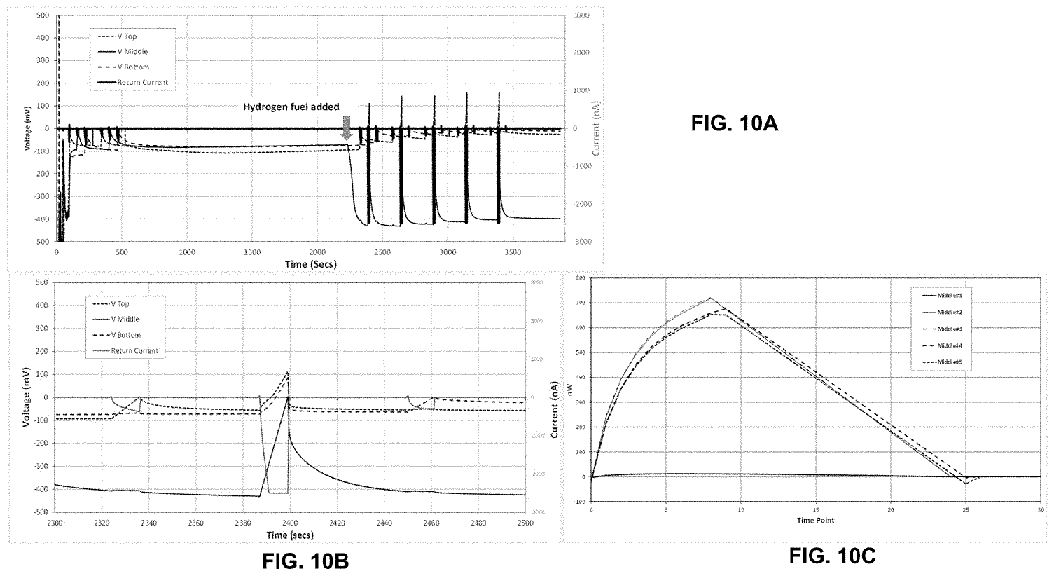

FIGS. 10A-C are graphs of the open circuit potentials and maximum power from electrodes using various kinds of ground electrodes. The catalyst was directly deposited on the working electrodes. The graphs also depict conditions with and without hydrogen as the fuel. The number of consecutive measurements are made serially on the same construct without removing the fcLFIA from the holder. Lower standard deviations imply a more constant signal vs. time. FIG. 10A is a plot of representative voltage and current curves for the three electrodes. At about 4500 seconds, hydrogen fuel is added and the voltage on the Middle electrode drops substantially as that is the electrode with catalyst present. FIG. 10B is an expansion of the current and voltage during stressing one of the three working electrodes by ramping the voltage as in Example 6. The open-circuit voltage reflects the impressed voltage by the D/A converter 93 and not the voltage generated by the fuel cells. Voltage and current readings are shown for the three electrodes with MnO₂ on the ground electrode. From the voltage and current measurements during the stress, power curves are calculated and shown in FIG. 10C. Only the curves from the Middle electrodes are shown as the others are similar to the Middle #1 measurement, where no fuel is present. The X-axis labels in FIG. 10C reflect the number of steps in the impressed voltage by the D/A converter voltage application and not the timing of the run. To make the data analysis easier, a constant number of steps are used (rather than a constant D/A step size) with the size of the steps calculated from the voltage span. The measurements were repeated five times for each electrode. Note, that often the current is opposite sign on electrodes that do not contain the catalyst. This provides more background discrimination. The hydrogen fuel is added at approximately 2300 seconds from the start by connecting a tube of flowing hydrogen to the side of the fcLFIA cartridge 63. A noticeable change in the voltage is observed.

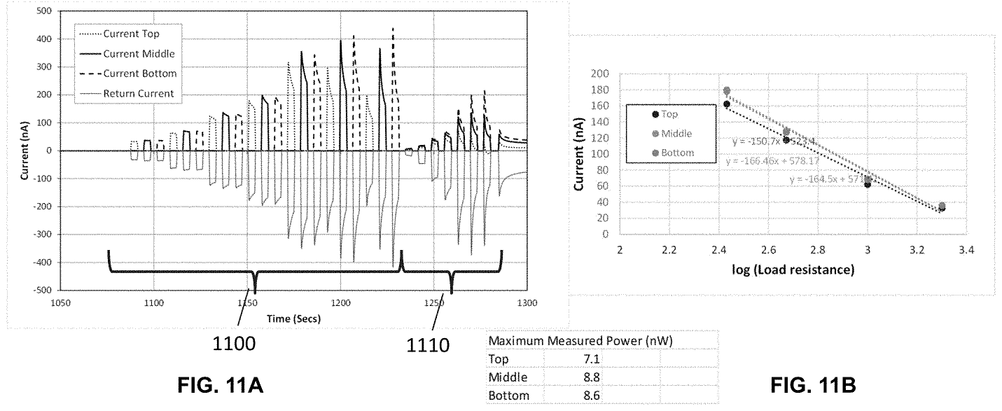

FIGS. 11A-B are graphs of the readings taken as outlined in Example 8 during stressing the fuel cell using load resistors. The electrode coupon was prepared as in Example 7. Only the data after hydrogen is added is shown. FIG. 11A is a graph of the currents during the stressing. In FIG. 11A the resistors are applied sequentially to each electrode and then the resistor values decreased to increase the load to generate data 1100. In FIG. 11A, the resistors are applied to each electrode simultaneously to generate data 1110. Also is plotted the return current which mirrors the current generated at each electrode showing that power is not generated in another location. FIG. 11B is a plot of log of the load resistance vs. measured average current for the first four loadings in 1100. The slopes are similar showing that each electrode has immobilize a similar amount of catalyst.

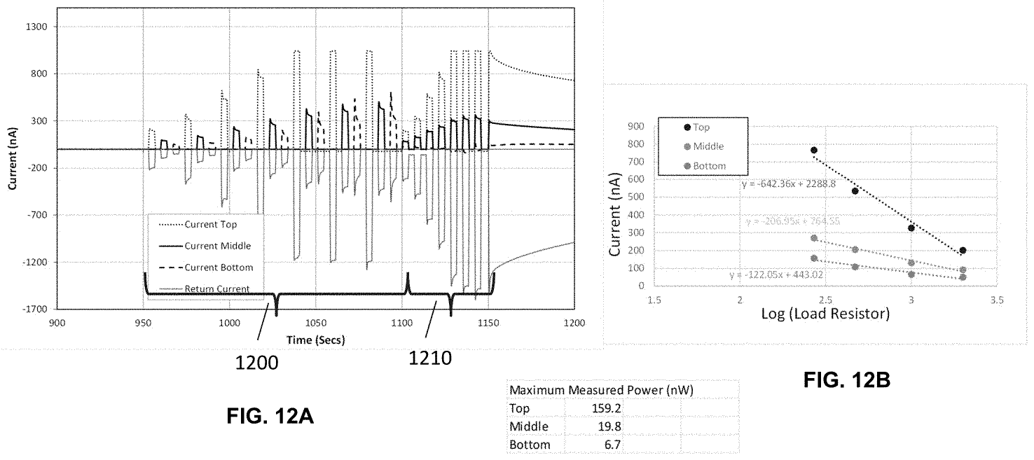

FIGS. 12A-B are graphs of the readings taken as outlined in Example 8 during stressing the fuel cell using load resistors. The electrode coupon was prepared as in Example 9. Only the data after hydrogen is added is shown. FIG. 12A is a graph of the currents during the stressing. In FIG. 12A, the resistors are applied sequentially to each electrode and then the resistor values decreased to increase the load to generate data 1200. In FIG. 12A, the resistors are applied to each electrode simultaneously to generate data 1210. Also is plotted the return current which mirrors the current generated at each electrode showing that power is not generated in another location. FIG. 12B is a plot of log of the load resistance vs. measured average current for the first four loadings in 1200. The slopes are different and reflect the varying amounts of catalyst on each electrode.

### DETAILED DESCRIPTION OF THE INVENTION

The present invention provides current and voltage proportional to the amount of catalyst immobilized on the electrodes through a biological binding event. In one embodiment, the electrode blanks are prepared as in Example 1. The binding elements can be placed as in Example 2 or Example 3 on the electrode blanks. The fcLFIA cartridge is assembled as in FIG. 4 with the prepared blanks. For analysis, the assembled fcLFIA is placed in a reader 55 that makes electrical contacts through the back. The test solution is then introduced into well 53. The test solution dissolves and mobilizes catalytic nanoparticles bound to biological binding entities. These nanoparticles are then captured on electrodes that contain the other pair of the binding pair forming a sandwich type assay. Alternatively, as known in the art, the binding of the initial binding pair to the analyte will affect the subsequent capture of the nanoparticle-analyte to the binding pair immobilized on the capture electrode. As the fluid moves across the electrodes, a voltage change is noted that can determine when fluid is present as depicted in FIG. 9. Typically a time elapse for all the fluid to flow before measurement occurs. However, unlike colorimetric LFIA, the fcLFIA does not require a clean background as only catalytic nanoparticles bound to the electrodes produce a current production in the presence of a fuel.

The fuel cell lateral flow immunoassay is a general term for a system that generates power though a chemical reaction. It may be a fuel cell where the components are not depleted and only fuel needs to be supplied or a battery where both the oxidant and reductant are depleted (sacrificial) or a hybrid where one of the oxidants or reductants is depleted (sacrificial) and the other is supplied remotely. In the fcLFIA, the catalytic nanoparticles are captured at much lower levels of less than 1 μg/cm² on the anode, when hydrogen is used as the fuel. Additionally, although no catalyst is necessary at the anode in the fcLFIA system, the presence of an easily oxidized material increases the power produced. The easily oxidized material is depleted in that embodiment.

The fcLFIA has some analogy to an electrochemical glucose sensor, which are well known in the art. As described in Yoo and Lee (Yoo and Lee 2010), glucose sensors use a set of enzymes that oxidize or reduce glucose to form higher energy compounds that can be detected electrochemically. One example is to use glucose oxidase, which catalyzes the oxidation of glucose with oxygen to form hydrogen peroxide. Another popular enzyme is glucose dehydrogenase that form NADH from NAD. To detect hydrogen peroxide or NADH, a potential is APPLIED to the electrodes such that at a given potential these molecules are either reduced or oxidized. Selectivity comes from the magnitude of the applied potential and timing. For example, the build-up of the high energy molecules is not instantaneous and during that time a background can be recorded. Because these sensors supply power, other molecules that have similar oxidation or reduction potentials will limit the sensitivity. Additionally, capacitance and conductivity of the media cause a background currents that limit the detection of the additional current from the presence of the high-energy molecule. Through judicious choice of label, other sensors have been developed that reduce interferences. For example, Guo and Yang, (Guo and Yang 2005) have developed sensors based on the oxidation of ruthenium tris(2,29-bipyridine), which is reduced back chemically with oxlate to complete the cycle. However, again they must supply power to cause the oxidation of the label and detection of the resultant current. The label may be either the oxidant or the reductant. In the instant invention, the assay provides power with the judicious choice of oxidant and reductant.

The measurements of pA currents is routinely done with modern electronics. Because of the proportionality constant between current and moles, 1 pA corresponds to approximately 1e-17 moles of a molecules undergoing a one electron change per second.

Fuel in the present invention is supplied either externally or more conveniently in an immobilized pad that gets activated by the flowing test fluid. Since the biology is complete after the fluid passes the electrodes, fuel generating materials can be incorporated in the absorption pad area. Additionally, the conditions in the absorption pad area need not be conducive to biological binding and may have extremes of pH or oxidation or corrosion. One preferred hydrogen generation material is sodium borohydride. This is a stable solid that readily produces hydrogen on contact with acids or water. As hydrogen is a gas, diffusion back to the electrode area can be rapid. Alternatively, the fuel can be generated electrochemically away from the biology and the fuel then diffuses to the biology to allow its detection.

Catalytic nanoparticles are desirable in the instant invention because they provide a current for a long time. However, they are not absolutely needed as electronics can make a reading rapidly. In that case, the nanoparticle could be the fuel rather than a catalyst. Metal-oxidant pairs for batteries are well known in the art. For example, immobilization of 1 ng of Zn nanoparticles would provide 1.5 E-11 moles of Zn to be oxidized to Zn⁺². This would provide over 20 nA of current for 100 seconds. Similar results would occur with other metals such as iron. The voltages and hence power of such “batteries” likely would be compromised because the system in the vicinity of the biology is at near neutral pH, room temperature, selective osmolality, and does not have a well engineered cell separator between the electrodes. In a battery, power and energy delivered to the load are important. However in the fcLFIA system, because current is what is needed to determine the mass or number of the nanoparticle labels and hence the amount of target analyte in the test solution, having the voltage optimized is not necessary.

The fcLFIA system lends itself to ready controls since numerous electrodes can be readily integrated. In the embodiment shown in FIG. 4, the four electrodes would comprise: one for ground, one electrode for a positive control (like in the prior art depicted in FIG. 1) one electrode for a negative control that measures background current, and one electrode for the analyte.

The nanoparticles are catalysts to electrical chemical events and may be prepared from various metals and metal oxides known in the art such as Pd, Pt, Ag, Fe, Ni, Rh, Rh, V, and Mo or mixtures thereof. The choice of catalytic particle depends on the type of fuel used to generate the power measured by the device. For hydrogen gas as the fuel, Pd and Pt nanoparticles are equally effective, with Pd nanoparticles preferred. For hydrogen peroxide as the fuel, MnO₂ nanoparticles are preferred. The nanoparticle need not be metals particles as iron chelates or other chelated metals may be employed as labels such as EDTA chelates of iron attached to the binding pair using chemistry known in the art. For iron as the catalyst and using hydrogen peroxide as the fuel, TAML (Collins and Gordon-Wylie 1998) is preferred.

Biological binding entities are known in the art. They may be antibodies, nucleic acids, proteins, or other molecules that can selectively bind some analyte. They need not be the same group of biological binding elements such as two antibodies or two strands of nucleic acid but could be an antibody paired with a nucleic acid. Alternatively, for small molecules or ions such as lead, the system may contain a molecule that selectively binds the ion and then that complex is recognized by the binding pair in the fcLFIA system.

Cartridge 63 may have writing or characters 66 that identify type of test to the user and to the reader upon insertion. For example, the writing may be a bar code or QR Codes read by the reader upon insertion. Alternatively or in conjuction, the cartridge 63 may have RFID tags embedded for identification of the cartridge and providing other information. Identification may comprise the type of assay i.e. for example what disease such as COVID-19, lot numbers, calibration information, date of manufacture, and other information useful in tracking the device from manufacture to use. The information and results of the test may be displayed to the user by the electronic reader or transmitted either wirelessly or through a cable to a remote facility for storage or analysis through methods known in the art. The transmitted data may be encrypted for privacy.

The ground electrode may be any easily oxidizable or reducible material. They may be coated alone or because the amount needed can be very small, coated over or mixed with the carbon electrode (such as sold by the Gwent Group, Code #C2030519P5 and Code #C2070424P2). They are chosen depending on the chemistry occurring at the working electrodes. For hydrogen oxidation at the working electrode due to the presence of a catalytic nanoparticle, some acceptable materials that are good electron acceptors are known in the art. Some examples are: bare carbon electrodes, Ag/AgCl, MnO₂, and dyes such as: Prussian Blue, Meldola Blue, Methylene Blue, Resorufin, Resazurin, 2,6-Dichloroindophenol, Phenazinium Methyl Sulfate, Indigo, Indigo carmine, or tetrazolium salts such as Nitrotetrazolium Blue. MnO₂ is preferred for hydrogen as the fuel. For labels that are reduced at the working electrode due to the presence of MnO₂ nanoparticles used as labels or H₂O₂ fuel in the presence of a catalytic nanoparticle, the ground electrode must be easily oxidized. Good electron donors are known in the art. Some examples are zinc, iron, aluminum, magnesium, Al/Mg alloys, or Prussian blue, and reduced forms of the dyes used as electron acceptors. Zinc is preferred for sacrificial (used in the battery mode of detection) MnO₂ nanoparticles.

### Example 1—Preparation of the Electrodes

For small-scale development, the electrodes may be made very conveniently by hand through a stencil process. A stencil material (Oratape MT80P) is placed on polycarbonate sheet (3M 665 PPC Film). The stencil is patterned with a computer-controlled, Silhouette Cameo 4 cutter and the appropriate holes weeded. The stencil is then coated with graphite ink (Ercon 3451, Ercon Incorporated, Wareham, Mass.), allowed to air dry or forced air dry, and the stencil overlay removed from the polycarbonate sheet providing a sheet of printed carbon electrodes. Ercon 3449 or Gwent C2030519P4 (Gwent Group, Pontypool, Gwent, UK) may be used in place of Ercon 3451 with equal results. These may be diced in a number of ways known in the art to allow deposition of the requisite chemistries. As this stenciling process is computer controlled, any change in the design can be made trivially. Selective area coating of inks are possible by weeding different areas of the stencil before applying the different inks. For large-scale manufacturing, the electrodes can be made by several processes known in the art such as screen printing or offset printing in roll format for easy assembly.

### Example 2—Preparation of Pd-Protein Catalyst

Streptavidin palladium conjugates were prepared by combining 9 μL of PdCl₂ (64 mM), 91 μL of Streptavidin (1 mg/mL in 90 mM phosphate buffer pH 8), and 242.5 μL of distilled water in a microfuge tube. The solution was equilibrated for 5 minutes before rapidly adding 15 μL sodium borohydride (10 mg/mL) with vigorous agitation. The solution immediately changed from a pale yellow to brown/gray color upon addition of the reductant. The microfuge tube was placed on a shaking table for a minimum of one hour to allow the reaction to go to completion. The palladium concentration was about 180 ng/μL.

### Example 3—Preparation of Pt-Protein Catalysts

Streptavidin platinum conjugates were prepared by combining 18 μL of PtCl₂ (64 mM), 182 μL of Streptavidin (2 mg/mL), and 970 μL of distilled water in a microfuge tube. The solution was equilibrated for 10 minutes before rapidly adding 30 μL sodium borohydride (10 mg/mL) with vigorous agitation. The solution immediately changed from a pale yellow to black color upon addition of the reductant. The microfuge tube was placed on a shaking table for a minimum of one hour to allow the reaction to go to completion. The platinum concentration was about 190 ng/μL.

### Example 4—Preparation of Manganese Dioxide Ground Electrodes

To the ground electrode (as defined in FIG. 8) of Ercon E3451 electrodes as prepared in Example 1, was placed 2 μL solution of KMnO₄ (1 mg/mL (6.3 mM)) in water followed by 2 μL solution of MnSO₄ monohydrate (10 mg/mL (59 mM)) in water. The water was allowed to evaporate at room temperature to form a brown film of MnO₂. Excess salts were removed with a brief water rinse.

### Example 5—Variation in Type of Ground Electrodes

To test which type of ground electrodes provided better performance, various types of oxidants in Table 1 (solids for the Fe₂O₃ and Fe₃O₄) or Example 4 for the MnO₂ were coated onto Ercon E3451 electrodes as prepared in Example 1 on the Ground electrode as defined in FIG. 8. Then, 2 μL of palladium catalyst prepared in Example 2 was directly deposited onto the Middle electrode as defined in FIG. 8 and air dried. The whole electrode assembly was briefly rinsed, air dried, and then dried at 60° C. before assembling into the fcLFIA holder as depicted in FIG. 3 and FIG. 4. The electrode assembly was inserted into the fcLFIA reader and 100 μL 100 mM phosphate buffer pH 8 added. Voltage and current measurements are continuously made on all three electrodes with the circuit outlined in FIG. 7 and FIG. 8 and recorded on a desktop computer at 1 group of samples/sec. Representative voltage and current curves are shown in FIG. 10A for the three electrodes with MnO2 on the Ground electrode. After stabilizing without fuel, the fuel cell is stressed as in Example 6. After some time, hydrogen gas is added to the side of the reader, the voltage increases, and when it stabilizes, the fuel cell is stressed as in Example 6, the current and voltage measured for each electrode, and power curves are calculated as in FIG. 10C. Stressing the fuel cell is repeated four times to determine the robustness of the signal. Averages of the maximum power from the electrode with the catalyst and the blanks are shown in Table 1.

TABLE 1
Current and Power with Various Types of Ground Electrodes

| Without Fuel - Average Power (nW) | | | | With Fuel - Average Power (nW) and Open-Circuit Voltage (mV) | | | | |
|---|---|---|---|---|---|---|---|---|
| | | | Electrode | | | Electrode | | Electrode |
| Type of | Blank | Standard | with | Blank | Standard | with | Standard | with |
| Ground | Electrodes | Deviation | Catalyst | Electrodes | Deviation | Catalyst | Deviation | Catalyst |
| Ercon | 0.27 | 0.38 | 12.41 | 3.22 | 2.54 | 96 | 20 | −163 |
| E3451 | | | | | | | | |
| carbon | | | | | | | | |
| MnO2 | 10.45 | 6.89 | 12.06 | 5.61 | 5.13 | 692 | 33 | −264 |
| Fe2O3 | 0.52 | 0.61 | 0.04 | 8.21 | 5.66 | 150 | 11 | −214 |
| Fe3O4 | 0.07 | 0.09 | 0.15 | 8.51 | 6.32 | 180 | 14 | −238 |

### Example 6—Stressing the Fuel Cell with the D/A

Referring to FIG. 8, in one embodiment, the fuel cell assembly of Example 5 is stressed by recording the open cell voltage (OCV). Then, a potential equal to the OCV is applied to the given electrode with the D/A 100 by closing switch 111 and having switch 112 open. Current is only measured in this configuration using the OP Amp 113 and the A/D 115 and the voltage on A/D 105. The voltage on D/A 100 is ramped from the OCV to ground and the current and voltage applied recorded. This is plotted with the desktop computer to generate plots shown in FIGS. 11A-B for each of the electrodes.

### Example 7—Demonstration of Approximately Equal Results for Equal Catalyst

The Streptavidin-platinum was diluted 1:100 and 2 uL applied to each of the working electrodes defined in FIG. 8. The electrodes were printed with Gwent carbon as outlined in Example 1. Ag/AgCl ink (Sigma-Aldrich Co. cat #901773) was applied over the carbon for the ground to provide better current, as outlined in Example 5. The electrode assembly was assembled into a fcLFIA holder (FIG. 4—63), 100 mM phosphate buffer pH 8 added, allowed to equilibrate, blank reading taken, and then the system exposed to hydrogen gas. The data was taken as in Example 8 and a snippet of the results displayed in FIGS. 11A-B. The slopes of the current-resistance curves are very similar indicating similar amounts of catalyst on each electrode.

### Example 8—Stressing the Fuel Cell with Load Resistors

Using the Electrodes as defined in FIG. 8, in one embodiment, the fuel cell assembly of Example 7 is stressed by applying load resistor 101 by closing switch 112 and having switch 111 open. Current is measured in this configuration using both OP Amp 113 with A/D 115 and OP Amp 103 with A/D 104. The voltage is also measured with A/D 105. Resistor 101 is decreased over a period of time. The electrodes may be measured simultaneously or individually by this method. The results are plotted with the desktop computer to generate plots shown in FIGS. 11A-B from the electrode assembly prepared in Example 7.

### Example 9—Demonstration of Quantitation

The Streptavidin-platinum was diluted 1:150, 1:100, and 1:200 and 2 uL applied to each of the working electrodes defined in FIG. 8. The electrodes were printed with Gwent carbon as outlined in Example 1. Ag/AgCl ink (Sigma-Aldrich Co. cat #901773) was applied over the carbon for the ground to provide better current, as outlined in Example 5. The electrode assembly was assembled into a fcLFIA holder (FIG. 4—63), 100 mM phosphate buffer pH 8 added, allowed to equilibrate, blank reading taken, and then the system exposed to hydrogen gas. The data was acquired as in Example 8 and a snippet of the results displayed in FIGS. 12A-B. The slopes of the current-resistance curves mirror the amounts of catalyst on each electrode.

### Example 10—Qualifying the Nanoparticle Conjugates

Slight changes to preparation of the protein-nanoparticle conjugates can affect the catalytic activity. Mulvaney, et al., qualified their nanoparticle conjugates using a colorimetric assay. Although one could expect that the electronic activity should parallel the chemical activity, the reactions are not the same. In the case of Mulvaney, et al., the chemistry is oxidation of a dye. In the instant invention, the chemistry is oxidation of a fuel. For electrical qualification, an appropriated diluted solution of the nanoparticle conjugates is placed on the top electrode 64 of the two electrode assembly in FIG. 4. Approximately 3.4 ng of each nanoparticle, as measured by the weight of the metal, was deposited on the test electrode. Manganese dioxide is deposited on the ground electrode as in Example 5. The electrode assemble is placed in a fcLFIA holder and run as in Example 5, stressed, and data taken as in Example 6. Results are shown in Table 2 for several preparations of nanoparticles. The power and voltage was an average over five stress cycles.

TABLE 2
Qualifying nanoparticle conjugates for electrical activity

| | | Open Circuit |
|---|---|---|
| Nanoparticle Conjugate | Max Power (nW) | Voltage (mV) |
| Pt-Streptavidin Preparation #1 | 254 | −342 |
| Pt-Streptavidin Preparation #2 | 346 | −357 |
| Pd-Streptavidin | 331 | −307 |
| Pd-Bovine Serum Albumin | 363 | −283 |

The above descriptions are those of the preferred embodiments of the invention. Various modifications and variations are possible in light of the above teachings without departing from the spirit and broader aspects of the invention. It is therefore to be understood that the claimed invention may be practiced otherwise than as specifically described. Any references to claim elements in the singular, for example, using the articles “a,” “an,” “the,” or “said,” is not to be construed as limiting the element to the singular.

What is claimed as new and desired to be protected by Letters Patent of the United States is:

1. A system for measuring the binding events in a lateral flow immunoassay, comprising:
an electrochemically active label bound to a first element of a binding pair;
an electrode bound to a second element of a binding pair, wherein the electrochemically active label is captured on the electrode to form an assay;
a ground electrode;
an electronic reading package for measuring current and voltage between the electrode and the ground electrode;
an electrochemically active fuel; and
a solution bridge between the electrode and the ground electrode;
wherein the assay produces the current between the electrode and the ground electrode in the presence of an electrochemically active fuel, and wherein the current is proportional to the amount of electrochemically active label immobilized on the electrode.

2. The system of claim 1, wherein the electrochemically active label comprises a catalyst that can oxidize or reduce the fuel.

3. The system in claim 1, wherein the electronic reading package quantifies the said current and voltage.

4. The system in claim 1, wherein the solution bridge comprises a liquid wetted membrane.

5. The system of claim 1 wherein the solution bridge comprises an aqueous solution.

6. The system of claim 1 wherein the ground electrode comprises an easily reducible material or an easily oxidizable material.

7. The system of claim 6, wherein the ground electrode comprises an easily reducible material, and the easily reducible material comprises a metal oxide, an organic molecule, an oxidized metal, or oxygen.

8. The system of claim 7, wherein the easily reducible material is MnO₂.

9. The system of claim 7, wherein the easily reducible material is silver chloride.

10. The system of claim 6, wherein the ground electrode comprises an easily oxidizable material, and the easily oxidizable material comprises a metal or an organic compound.

11. The system of claim 10, wherein the easily oxidizable material is zinc.

12. The system of claim 10, wherein the easily oxidizable material is iron.

13. A method for measuring the binding events in a lateral flow immunoassay, comprising:
introducing a test solution to a lateral flow immunoassay, wherein the test solution mobilizes an electrochemically active label bound to a first element of a binding pair;
capturing the electrochemically active label on an electrode containing a second element of the binding pair to form an assay;
measuring a current and voltage between the electrode and a ground electrode that occurs as the test solution moves across the electrode; and
providing an electrochemically active fuel; wherein the assay produces the current between the electrode and the ground electrode in the presence of an electrochemically active fuel, and wherein the current is proportional to the amount of electrochemically active label immobilized on the electrode.

14. The method of claim 13, wherein the electrochemically active label comprises a catalyst that can oxidize or reduce the fuel.

15. The method of claim 13, additionally comprising quantifying the current and voltage.

16. The method of claim 13, wherein the ground electrode comprises an easily reducible material or an easily oxidizable material.

17. The method of claim 16, wherein the ground electrode comprises an easily reducible material, and the easily reducible material comprises a metal oxide, an organic molecule, an oxidized metal, or oxygen.

18. The method of claim 17, wherein the easily reducible material is MnO₂.

19. The method of claim 17, wherein the easily reducible material is silver chloride.

20. The method of claim 16, wherein the ground electrode comprises an easily oxidizable material, and the easily oxidizable material comprises a metal or an organic compound.

21. The method of claim 20, wherein the easily oxidizable material is zinc.

22. The method of claim 20, wherein the easily oxidizable material is iron.
